(19) United States
(12) Patent Application Publication
(10) Pub. No.: US 2018/0184689 A1
(43) Pub. Date: Jul. 5, 2018

(54) A METHOD FOR PREPARING LUTEIN FEED ADDITIVE

(71) Applicants: Zhejiang New Weipu Additive Co., Ltd., Shaoxing, Zhejiang (CN); NINGBO UNIVERSITY OF TECHNOLOGY, NINGBO, ZHEJIANG PROVINCE (CN); ZHEJIANG NHU COMPANY LTD., SHAOXING, ZHEJIANG PROVINCE (CN); ZHEJIANG UNIVERSITY, HANGZHOU, Zhejiang Province (CN)

(72) Inventors: DAN QIU, NINGBO, ZHEJIANG PROVINCE (CN); ZHIRONG CHEN, HANGZHOU, ZHEJIANG PROVINCE (CN); LIFANG SHI, SHAOXING, ZHEJIANG PROVINCE (CN); JIANDONG LI, SHAOXING, ZHEJIANG PROVINCE (CN); LINPU CAI, SHAOXING, ZHEJIANG PROVINCE (CN); YUFENG LIANG, SHAOXING, ZHEJIANG PROVINCE (CN); MINCONG CAI, SHAOXING, ZHEJIANG PROVINCE (CN); LIFENG ZHAO, SHAOXING, ZHEJIANG PROVINCE (CN)

(21) Appl. No.: 15/740,777

(22) PCT Filed: Jun. 21, 2016
(86) PCT No.: PCT/CN2016/086498

§ 371 Date: Dec. 28, 2017

(30) Foreign Application Priority Data
Jul. 9, 2015 (CN) 201510405640.5

(51) Int. Cl. A23K 40/35; A23K 20/179; C07C 403/24

(52) U.S. Cl. CPC A23K 40/35; C07C 2601/16; C07C 403/24; A23K 20/179

(57) ABSTRACT

The present invention discloses a method for preparing controlled release type lutein feed additive. Steps: lutein extract, glycerol and fat are mixed and dissolved, then alkali liquor is dropped under condition of mechanical stirring and sheering emulsification to allow the lutein to be fully saponified. Then fat is added and alkaline saponification is conducted, adjusting the viscosity and neutralizing and conducting spray granulation to the mixed liquor to obtain controlled release lutein feed additive. By utilizing the combined effects of glycerol and fat, the present invention realizes saponification and microcapsule formation using simple method. Comparing to conventional absorption processes, the present invention effectively improves the chemical stability of lutein in light, heating and acid environment, allowing the controlled release type lutein feed additive product to barely release in gastric fluid environment but fully release in intestinal fluid environment, hence achieving higher bioavailability.

### TECHNICAL FIELD

The present invention relates to the feed additive technical field, specifically relates to a method for preparing lutein feed additive.

### BACKGROUND ART

Carotenoids widely exist in the nature, of which the most important representatives are β-carotene, astaxanthin, lycopene, canthaxanthin and lutein etc. Luteins widely exist in vegetables, flowers, fruits and some Algae. Lutein is widely used, wherein in the feed field, it is added to chicken feed as a natural colorant to improve the colors of the egg yolk and chicken skin. Therefore, extracting and separating lutein from natural plants such as marigold and calendula has gradually become a research hotspot, more and more enterprises are engaging in this industry. However, lutein obtained from extracting plants generally exists in the extract in the form of fatty acid ester, and lutein ester cannot be directly absorbed and utilized by human and poultry. Only after it is saponified into free lutein, can it be used.

Currently, there are many reported methods for preparing lutein products. However, major methods for preparing feed additives using the extract as the raw material are as follows:

CN101433266A reports a method for preparing feed additive containing natural free-state lutein. In this method, lutein extract of no less than 15% content is mixed with saturated potassium hydroxide methanol solution and is saponified for 2-8 hours, and then is mechanically mixed with free-flowing agents, such as calcium silicate, and ingredient carriers, such as corncob powder, at various proportions to obtain feed additive containing free-state lutein.

CN101473893A discloses a method for preparing feed additive containing lutein and zeaxanthine, comprising: mixing lutein ester with propylene glycol and alkali liquor to react under 70-120° C. for 2-8 hours to obtain mixed lutein-zeaxanthine paste, and finally mechanically mixing the lutein-zeaxanthine paste with free-flowing agents, such as calcium silicate, and ingredient carriers, such as corncob powder, at various proportions to obtain feed additive. The difference between this method and CN101433266A is that the step for converting partial lutein into zeaxanthine is added, other processes are basically the same. It is obvious that high temperature treatment can lead to isomerism and degradation occurred to carotenoids such as lutein etc., resulting in reduced biological value.

CN103058906A also introduces a technology for producing lutein feed additive, in which propylene glycol, potassium hydroxide solution and antioxidant are added to calendula extract to obtain saponification solution, then silicon dioxide powder is added to obtain lutein feed additive through stirring and screening.

The commonality of the above 3 technologies is in that: firstly, lutein extract is placed in alkaline solvent environment, lutein ester is saponified and converted into free lutein, then the saponified mixed liquor is mixed with a mineral absorbent while the solvent is recovered, the effective substance of lutein is absorbed on the surface and micropore structure of the mineral absorbent. Due to that lutein is highly sensitive to light, heating and acid environment (such as the gastric fluid environment in living body), lutein exposed on the surface of mineral absorbent is very easy to deteriorate, meanwhile, lutein absorbed into the micropore structure of the mineral absorbent is difficult to fully release in living body, resulting in reduced bioavailability.

There are also many reports on application of lutein products in other fields, including: CN 1757315A: lutein ester is heated and liquefied under 30-40° C. condition, then is saponified, emulsified and homogenized to obtain lactescence product; CN 101292950A: carotenoids and polymers such as polyethylene glycol are melted together under high temperature, then additive is added, mixed and cooled to obtain carotenoid solid dispersion; CN 101836954B: lutein powder and fat are mixed and go through high pressure homogenization to obtain oil suspension; CN 101869261B: lutein ester is mixed and heated with plant oil, then add hydrated agent containing Tween and Span, finally alcohol diluent is added to obtain water-soluble lutein ester; CN 102949349A: lutein active pharmaceutical ingredients and ethyl cellulose are dissolved in ethyl acetate, then are added to water phase containing Tween and ethyl acetate, and are emulsified and further diluted by adding water, and are diffused using ethyl acetate, allowing the emulsion droplet to be solidified and separated out; CN 103637022A: lutein crystals are dispersed into huge amount of organic solvents such as ethyl alcohol, then surfactants such as Tween etc. and silica aerogel are added to obtain solid powder, then carriers such as calcium carbonate are added for dilution to produce water-soluble modified lutein.

For all of the above mentioned methods, their raw materials are high purity lutein or lutein ester, which is far different from extract, because extract cannot be melted and is difficult to solve, of which the complex composition results in its special physical property; meanwhile, the raw materials or processes used by such methods have high requirements, and it is hardly possible to be used as feed additive.

### SUMMARY OF THE INVENTION

The present invention is to overcome the shortcomings of the above mentioned prior arts and provide a method for preparing lutein feed additive using lutein extract as a raw material, the lutein feed additive produced using this method has higher stability, and barely releases in gastric fluid environment but fully releases in intestinal fluid environment.

A method for preparing lutein feed additive, comprising the following steps:

(1) mixing and heating lutein extract, glycerol, fat and antioxidant until they are dissolved into each other to form oil phase;

(2) Under high speed sheering condition, dropping alkali liquor into the oil phase obtained in Step (1), then stirring at low speed to continue saponification;

(3) After the lutein ester is fully saponified, supplement fat and stirring to form lutein fat solution;

(4) Adding alkali liquor into the lutein fat solution obtained in Step (3), heating to react until the viscosity reaches 300˜600 cp to obtain lutein mixed liquor;

(5) Adding acid to the lutein mixed liquor obtained in Step (4) to neutralize until pH7-8, then conducting fluidized bed spray granulation while it is still hot to form microcapsule of which the surface is wrapped by starch granules, which is dried to obtain the lutein feed additive.

Comparing to the prior art, in the process for preparing lutein feed additive, the present invention abandons the conventional absorption technology, and proposes for the first time to introduce glycerol and fat during saponification of lutein extract. Before saponification, glycerol and fat can help to decompose and dissolve lutein extract, during saponification, fatty acid salt produced by glycerol and fat can be used as an emulsifier to stabilize both the water and oil phases. After saponification is completed, the supplemented fat and its saponification product can regulate the softening point of the final product. During spray granulation, the saponification product of the fat and the high-melting-point ingredients in the extract together become the excipient of the microcapsule core, not only assisting product formation, but also providing tight protection layer for the lutein. Quite a portion of the lutein product produced from conventional absorption process is exposed on the surface of the absorbent, of which the chemical stability is far behind that of the product produced from the present invention.

In addition, when the lutein feed additive produced by the present invention enters the living body, in the strong acid gastric fluid environment, the existence of huge amount of fat acid that is difficult to dissolve in water insulates the destruction effect of the acid to the lutein. Whereas when the product enters the neutral intestinal fluid environment, the fat acid changes into fatty acid salt that can dissolve in water, resulting in full release of lutein, hence release control is achieved, barely with release in gastric fluid environment and with fully release in intestinal fluid environment. As to the lutein product produced from conventional absorption technology, a proportion of it is absorbed to the micropore structure of the absorbent, resulting in that such proportion of lutein is difficult to release, hence the bioavailability is lower than that of the lutein product produced from the present invention.

In the present invention, the fat is common plant oils and animal oils such as soybean oil, corn oil, peanut oil, coconut oil, sesame oil, olive oil, palm oil, colza oil, rice bran oil, sunflower seed oil, cocoa oil, butterfly, lard, beef tallow or mixture of the same;

The fats in Step (1) and Step (3) can be the same or different.

Preferably, the fat is at least one of the palm oil, lard and beef tallow. Under this condition, both the stability and bioavailability of the obtained lutein feed additive are higher.

In the present invention, no need to use high purity lutein or lutein ester as the raw material, and the content of lutein extract is measured by using method provided by GB 26405-2011. Preferably, the mass percent content of the lutein extract is about 15%, wherein lutein extract of about 15% content is the lutein extract that is commonly used in the industry, which is market sale product, of which the content can fluctuate to a certain extent.

In Step (1), the mass ratio of lutein extract, glycerol, fat and antioxidant is 100:10˜100:30˜50:0.1˜1;

In Step (3), the mass ratio of the supplemented fat to lutein extract is 50˜100:100.

Preferably, in Step (1), the antioxidant is tocopherol, BHT, TBHQ or ethoxyquin.

In Step (1), the heating temperature is 40˜60° C. at heating duration of 3˜10 minutes.

In Step (2), the low speed stirring rate is 100˜500 rpm, and the saponification temperature is 40˜80° C.

In Step (2), the saponification degree of the lutein ester is measured by High Performance Liquid Chromatography, when the normalization percentage of the peak area of the lutein liquid phase is >97%, it is deemed as complete saponification.

In the present invention, the alkali liquor is water solution of sodium hydroxide or potassium hydroxide of 30%˜50% mass concentration.

Wherein the alkali liquor added in Step (2) is primarily to saponify the luein extract, the alkali liquor added in Step (4) is primarily to saponify the fat, and the mass ratio of the alkali liquor added in Step (2) to the lutein extract is 30˜60:100;

In Step (4), the mass ratio of the alkali liquor to lutein extract is 10˜50:1.

In Step (4) in the present invention, the heating temperature is 40˜80° C.

Preferably, in Step (5), the used acid is hydrochloric acid of 10%˜20% mass percent concentration.

The lutein content of the lutein feed additive obtained from the present invention is measured using method provided by GB/T 21517-2008.

Based on the above analysis, by utilizing the combined effects of glycerol and fat, the present invention successfully realizes saponification and microcapsule formation using simple method, providing effective protection layer for the lutein. The present invention improves the chemical stablity of lutein in light, heating and acid environment, allowing the controlled release type lutein feed additive product to barely release in gastric fluid environment but fully release in intestinal fluid environment, hence achieving higher bioavailability, distinctive from the prior art.

### BRIEF DESCRIPTION OF THE DRAWINGS

FIG. 1 is pre-post-saponification High Performance Liquid Chromatography of the lutein extract in Embodiment 1;

FIG. 2(a) is the scanning electron microscope image of 2 lutein feed additive samples produced from Embodiment 1; FIG. 2(b) is the scanning electron microscope image of the lutein feed additive sample produced from comparison example 1.

FIG. 3(a) is the content change graph of 2 lutein feed additive samples produced from Embodiment 1 (A) and comparison example (B) under natural light illumination;

FIG. 3(b) is the content change graph of 2 lutein feed additive samples produced from Embodiment 1 (A) and comparison example (B) under 90° C.

### SPECIFIC EMBODIMENTS OF THE INVENTION

Below is further description of the present invention in combination with Specification figures and specific implementation.

### Embodiment 1

1) 100 g lutein extract (15.8 wt % of lutein content), 10 g glycerol, 50 g coconut oil and 1 g ethoxyquin were mixed and heated under 60° C. for 3 minutes and dissolved into an oil phase;

2) 30 g potassium hydroxide water solution of 50% mass concentration was dropped into the oil phase obtained in Step 1), and the high speed sheering rate was controlled at 10000 rpm, after the alkali liquor dropping was completed, high speed sheering was stopped, stirring was performed at 500 rpm to continue saponification, during the whole process the temperature was controlled at 80° C.;

3) After HPLC detected that the lutein saponification percentage had exceeded 97%, 100 g beef tallow was added continuously, stirring was performed at 500 rpm to dissolve into a lutein fat solution;

4) 50 g sodium hydroxide water solution of 30% mass concentration was dropped into the lutein fat solution obtained from Step 3), the temperature during the whole process was controlled at 80° C., and the mechanical stirring rate was 500 rpm, after the alkali liquor dropping was completed, the viscosity was allowed to reach 600 cp through thermal reaction to obtain lutein mixed liquor;

5) Hydrochloric acid of 20% mass concentration was used to neutralize the lutein mixed liquor obtained in Step (4) to pH7, then fluidized bed spray granulation was conducted while it was still hot to form microcapsule of which the surface was wrapped by starch granules, which was dried to obtain controlled release lutein feed additive product.

Based on test, the lutein content of such lutein feed additive product was 4.21%.

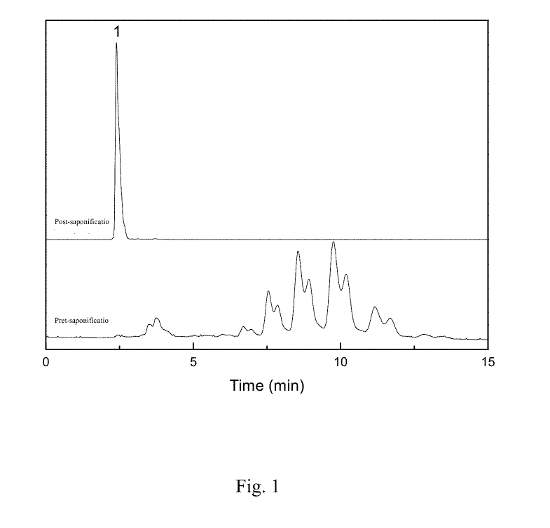

FIG. 1 is pre-post-saponification High Performance Liquid Chromatography of the lutein extract in Embodiment 1 (Peak No. 1 is lutein, the chromatographic peaks with duration of 2.5˜12.5 min are different lutein esters), the matographic column of HPLC is Agilent column C18, with methyl alcohol:acetonitrile:dichloromethane=2:1:2 as flowing phase at flow rate of 1 ml/min, the column temperature is 30° C. and it is measured under 473 nm.

### Comparison Example 1

Lutein feed additive product absorbed by mineral absorbent was prepared by adopting the patented technology of CN103058906A. This application holds the view that product produced from this comparison example can reflect the typical characteristics of the patented technologies of CN101433266A, CN101473893A and CN103058906A.

1) 100 g lutein extract (15.8 wt % of lutein content), 60 g propylene glycol and 3 g ethoxyquin were evenly mixed under 60° C.;

2) 50 g potassium hydroxide water solution (50% mass percent concentration) was added and stirred at 100 rpm, lutein saponification was completed as measured after 1 hour, lutein mixed liquor was obtained;

3) Then lutein mixed liquor of 60° C. was added to 230 g absorbent (calcium carbonate:silicon dioxide=4:6) of 90° C., absorbent was continuously stirred at constant 90° C. to allow the lutein to be absorbed into the absorbent, and the evaporated water and solvent were recovered;

4) decompression and desolvation were further conducted to ensure the residual solvent of the lutein feed additive was below the limit of detection, then screened to obtain lutein feed additive.

Based on test, the lutein content of such lutein feed additive was 4.32%.

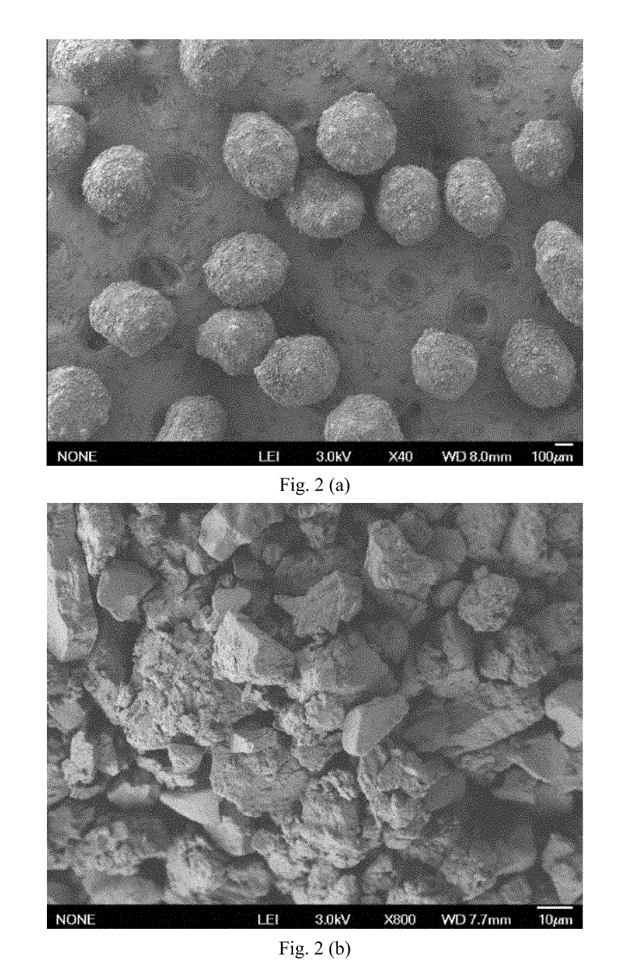

FIG. 2 is the scanning electron microscope image of 2 lutein feed additive samples produced from Embodiment 1 (FIG. a) and comparison example (FIG. b). It showed that the lutein additives produced from Embodiment 1 are microcapsules wrapped by explicit investment material with 100˜200 μm diameters, whereas the lutein additives produced from comparison example have uneven diameters up to tens of and are different degree of agglomerates by the mineral absorbent.

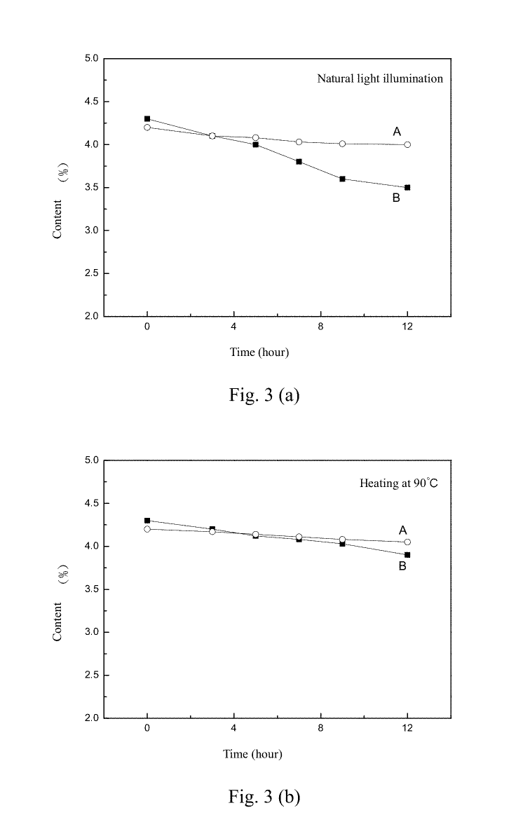

FIG. 3 is the content change graph of 2 lutein feed additive samples produced from Embodiment 1 (A) and comparison example (B) under natural light illumination and 90° C.; Abscissa (x-coordinate) is time, ordinate (y-coordinate) is lutein content in the sample as determined by ultraviolet spectroscopy. Curve A represents lutein feed additive produced from Embodiment 1, curve B represents lutein feed additive produced from comparison example. Wherein FIG. 3 (a) is the lutein content change graph after A and B are exposed to natural light for 12 hours (6:00˜18:00), content of A decreases from 4.21% to 4.02%, content of B decreases from 4.32% to 3.53%, indicating that the photostability of the lutein feed additive produced from Embodiment 1 is obviously better than that of the comparison example. FIG. 3 (b) is lutein content change graph after A and B are placed in drying oven of 90° C. heating for 12 hours, the content of A decreases from 4.21% to 4.05%, the content of B decreases from 4.32% to 3.91%, indicating that the thermal stability of the lutein feed additive produced from Embodiment 1 is also better than the comparison example.

### Embodiment 2

1) 100 kg lutein extract (15.2 wt % of lutein content), 100 kg glycerol, 30 kg soybean oil and 0.1 kg BHT were mixed and heated under 40° C. for 10 minutes to be dissolved into oil phase;

2) 60 kg potassium hydroxide of 40% mass concentration was dropped into the oil phase obtained in Step 1), and the high speed sheering rate was controlled at 3000 rpm, after the alkali liquor dropping was completed, high speed sheering was stopped, stirring was conducted at 100 rpm to continue saponification, during the whole process the temperature was controlled at 40° C.;

3) After HPLC detected that the lutein saponification percentage had exceeded 97%, 50 kg palm oil was continuously added, stirring as conducted at 200 rpm to dissolve into lutein fat solution;

4) 10 kg potassium hydroxide of 45% mass concentration was dropped into the lutein fat solution obtained from Step 3), the temperature during the whole process was controlled at 40° C., and the mechanical stirring rate was 100 rpm, after the alkali liquor dropping was completed, the viscosity was allowed to reach 300 cp through thermal reaction to obtain a lutein mixed liquor;

5) Hydrochloric acid of 10% mass concentration was used to neutralize the lutein mixed liquor obtained in Step (4) to pH8, then fluidized bed spray granulation was conducted while it was still hot to form microcapsule of which the surface was wrapped by starch granules, which was dried to obtain controlled release lutein feed additive product.

Based on test, the lutein content of such lutein feed additive was 3.96%.

### Embodiment 3

1) 100 kg lutein extract (15.7 wt % of lutein content), 80 kg glycerol, 40 kg corn oil and 0.5 kg tocopherol were mixed and heated under 50° C. for 5 minutes to be dissolved into an oil phase;

2) 40 kg sodium hydroxide of 42% mass concentration was dropped into the oil phase obtained in Step 1), and the high speed sheering rate was controlled at 5000 rpm. After the alkali liquor dropping was completed, high speed sheering was stopped, stirring was conducted at 300 rpm to continue saponification, during the whole process the temperature was controlled at 60° C.;

3) After HPLC had detected that the lutein saponification percentage had exceeded 97%, 40 kg lard was continuously added, stirring was conducted at 250 rpm to dissolve into a lutein fat solution;

4) 20 kg sodium hydroxide of 38% mass concentration was dropped into the lutein fat solution obtained from Step 3), the temperature during the whole process was controlled at 70 C, and the mechanical stirring rate was 400 rpm, after the alkali liquor dropping was completed, the viscosity was allowed to reach 500 cp through thermal reaction to obtain a lutein mixed liquor;

5) Hydrochloric acid of 15% mass concentration was used to neutralize the lutein mixed liquor obtained in Step (4) to pH7.5, then fluidized bed spray granulation was conducted while it was still hot to form microcapsule of which the surface was wrapped by starch granules, which was dried to obtain controlled release lutein feed additive product.

Based on test, the lutein content of such lutein feed additive was 4.52%.

### Embodiment 4

1) 100 kg lutein extract (15.5 wt % of lutein content), 10 kg glycerol, 17 kg cocoa oil, 15 kg colza oil and 0.8 kg TBHQ (tert-butyl hydroquinone) were mixed and heated under 45° C. for 7 minutes to be dissolved into oil phase;

2) 45 kg potassium hydroxide of 50% mass concentration was dropped into the oil phase obtained in Step 1), and the high speed sheering rate was controlled at 6000 rpm, after the alkali liquor dropping was completed, high speed sheering was stopped, stirring was conducted at 400 rpm to continue saponification, during the whole process the temperature was controlled at 65° C.;

3) After HPLC had detected that the lutein saponification percentage had exceeded 97%, 9 kg butterfly and 1 kg sesame oil were continuously added, stirring was conducted at 100 rpm to dissolve into a lutein fat solution;

4) 12 kg sodium hydroxide of 50% mass concentration was dropped into the lutein fat solution obtained from Step 3), the temperature during the whole process was controlled at 70° C., and the mechanical stirring rate was 450 rpm, after the alkali liquor dropping was completed, the viscosity was allowed to reach 480 cp through thermal reaction to obtain a lutein mixed liquor;

5) Hydrochloric acid of 15% mass concentration was used to neutralize the lutein mixed liquor obtained in Step (4) to pH7.2, then fluidized bed spray granulation was conducted while it was still hot to form microcapsule of which the surface was wrapped by starch granules, which was dried to obtain controlled release lutein feed additive product.

Based on test, the lutein content of such lutein feed additive was 6.86%.

### Embodiment 5

1) 100 g lutein extract (15.0 wt % of lutein content), 100 g glycerol, 38 g rice bran oil, 10 g sunflower seed oil and 0.9 g ethoxyquin were mixed and heated under 55° C. for 8 minutes to be dissolved into an oil phase;

2) D57 g sodium hydroxide of 39% mass concentration was dropped into the oil phase obtained in Step 1), and the high speed sheering rate was controlled at 7000 rpm. After the alkali liquor dropping was completed, high speed sheering was stopped, stirring was conducted at 450 rpm to continue saponification, during the whole process the temperature was controlled at 60° C.;

3) After HPLC had detected that the lutein saponification percentage had exceeded 97%, 50 g olive oil and 50 g peanut oil were continuously added, stirring was conducted at 180 rpm to dissolve into a lutein fat solution;

4) 48 g potassium hydroxide of 50% mass concentration was dropped into the lutein fat solution obtained from Step 3), the temperature during the whole process was controlled at 65° C., and the mechanical stirring rate was 400 rpm, after the alkali liquor dropping was completed, the viscosity was allowed to reach 530 cp through thermal reaction to obtain a lutein mixed liquor;

5) Hydrochloric acid of 15% mass concentration was used to neutralize the lutein mixed liquor obtained in Step (4) to pH7.7, then fluidized bed spray granulation was conducted while it was still hot to form microcapsule of which the surface was wrapped by starch granules, which was dried to obtain controlled release lutein feed additive product.

Based on test, the lutein content of such lutein feed additive was 3.02%.

### In Vitro Release Test Comparison:

The e sample produced from Embodiment 1 (sample A with 4.21% of lutein content) and the sample produced from comparison example (sample B with 4.32% of lutein content) were taken to conduct in vitro release test comparison.

1 g of samples were added into 100 ml simulated gastric fluids, stirring was conducted under 37° C. at 100 rpm for 0.5 and 1 hour, the dissolution rates of the luteins were measured respectively. The above mentioned solutions were kept static for 5 minutes, then pH was adjusted to 7.0, and 400 ml simulated intestinal fluid was added to stir under 37° C. at 100 rpm, the dissolution rate of the luteins was measured every 1 hour.

Lutein dissolution rate=lutein content of solution/total lutein content of sample

Wherein, the lutein content of the solution was quantified by an external standard method by taking partial test solution to centrifuge and the supernatant was tested using HPLC.

The actual in vitro release results are as shown in Table 1.

TABLE 1
Dissolution rates of different lutein feed additives

| | Gastric | Gastric | Intestinal | Intestinal | Intestinal |
|---|---|---|---|---|---|
| | fluid 0.5 h | fluid 1 h | fluid 1 h | fluid 3 h | fluid 5 h |
| Sample A | 9.08% | 11.41% | 66.47% | 83.52% | 96.17% |
| Sample B | 29.61% | 49.02% | 67.56% | 66.84% | 67.13% |

Results in Table 1 showed that lutein feed additive A produced by the present invention released only 11.41% of lutein in gastric fluid environment for 1 hour, and subsequently almost fully released the lutein after 5 hours in an intestinal environment. This was possibly due to that, in the strong acid environment in gastric fluid, most of the fat saponification products of A exist in the form of fatty acid, and long carbon chain fatty acid is insolvable in water, resulting in that the lutein in product A is very difficult to release in gastric fluid environment, whereas in the moderate intestinal fluid environment, the fatty acid in product A changes into fatty acid salt, resulting in that the lutein in product A can release slowly in intestinal fluid environment.

The lutein feed additive B produced from comparison example released 49.02% of lutein after 1 hour in gastric fluid environment, and subsequently reached absorption balance in an intestinal fluid environment within a short time, only about 67% of lutein can be released at most. This indicated that about 33% of effective substance of lutein was left in the absorption material due to the micropore structure of the mineral absorption material.

1. A method for preparing lutein feed additive, which is characterized in comprising the following steps:
(1) mixing lutein extract, glycerol, fat and antioxidant and heating until they are dissolved into each other to form an oil phase;
(2) under high speed sheering condition, dropping alkali liquor into the oil phase obtained in Step (1), then stirring at a low speed to continue saponification; a high speed sheering rate being 3000˜10000 rpm;
(3) after the lutein ester being fully saponified, supplementing fat and stirring to form a lutein fat solution;
(4) adding an alkali liquor into the lutein fat solution obtained in Step (3), heating to react until the viscosity reaches 300˜600 cp to obtain a lutein mixed liquor;
(5) adding acid to the lutein mixed liquor obtained in Step (4) to adjust pH to 7-8, then conducting fluidized bed spray granulation while still being hot to form microcapsule of which microcapsule's surface is wrapped by starch granules, which is dried to obtain the lutein feed additive.

2. The method for preparing lutein feed additive according to claim 1, which is characterized in that: the fat is soybean oil, corn oil, peanut oil, coconut oil, sesame oil, olive oil, palm oil, colza oil, rice bran oil, sunflower seed oil, cocoa oil, butterfly, lard, beef tallow or mixture of the same;
the fats in Step (1) and Step (3) can be the same or different.

3. The method for preparing lutein feed additive according to claim 1, which is characterized in that: the fat is at least one of the palm oil, lard and beef tallow;

4. The method for preparing lutein feed additive according to claim 1, which is characterized in that: in Step (1), the mass percent content of the lutein extract is about 15%, and the mass ratio of lutein extract, glycerol, fat and antioxidant is 100:10˜100:30˜50:0.1˜1;
in Step (3), the mass ratio of the supplemented fat to lutein extract is 50˜100:100.

5. The method for preparing lutein feed additive according to claim 1, which is characterized in that: in Step (1), the antioxidant is tocopherol, BHT, TBHQ or ethoxyquin.

6. The method for preparing lutein feed additive according to claim 1, which is characterized in that: in Step (2), the low speed stirring rate is 100˜500 rpm, and the saponification temperature is 40˜80° C.

7. The method for preparing lutein feed additive according to claim 1, which is characterized in that: in Step (2), the saponification degree of the lutein ester is measured by High Performance Liquid Chromatography, when the normalization percentage of the peak area of the lutein liquid phase is >97%, which is deemed as complete saponification.

8. The method for preparing lutein feed additive according to claim 1, which is characterized in that: the alkali liquor is water solution of sodium hydroxide or potassium hydroxide of 30%˜50% mass concentration;
wherein in Step (2), the mass ratio of the alkali liquor to lutein extract is 30˜60:100; and
wherein in Step (4), the mass ratio of the alkali liquor to lutein extract is 10˜50:1.

9. The method for preparing lutein feed additive according to claim 1, which is characterized in that: in Step (4), the heating temperature is 40˜80° C.

10. The method for preparing lutein feed additive according to claim 1, which is characterized in that: in Step (5), the used acid is hydrochloric acid of 10%˜20% mass percent concentration.
